(19) United States
(12) Patent Application Publication
(10) Pub. No.: US 2012/0116276 A1
(43) Pub. Date: May 10, 2012

(54) EXPANSIBLE DEVICE OF MEDICAL USE FOR SPECIFIC COMPRESSION OF THORAX, SYSTEM COMPRISING IT AND METHOD FOR CORRECTING A THORACIC DEFORMITY IN A PATIENT

(76) Inventors: Marcelo Hernan Martinez Ferro, Buenos Aires (AR); Carlos Pablo Fraire, Ciudad Autonoma de Bs. As (AR)

(21) Appl. No.: 13/352,803

(22) Filed: Jan. 18, 2012

Related U.S. Application Data
(62) Division of application No. 12/511,817, filed on Jul. 29, 2009.

(51) Int. Cl. A61F 5/00

(52) U.S. Cl. 602/19

(57) ABSTRACT

An expansible device of medical use for thorax specific compression including an applicable framework as a belt around a patient's thorax. The belt has a front arc and a back arc faced by concave inner sides and connected on both sides of the thorax by a hinge and by an adjustable side closure. The front arc includes a plate that compresses the deformity, and adjustable suspension for keeping both arcs spaced hanged from the shoulders. The belt also includes at least six joined bent plates, each arc being formed by at least three plates the ends of which overlap connecting the plates by a fixer. The compressing plate includes anchorages for associating an equipment for measuring the pressure made in adjusting the closure between the arcs. The invention includes a device, system, and method to treat a thoracic deformity.

### CROSS-REFERENCE TO RELATED U.S. APPLICATIONS

Not applicable.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

### REFERENCE TO AN APPENDIX SUBMITTED ON COMPACT DISC

Not applicable.

### BACKGROUND OF THE INVENTION

1. Field of the Invention

The present invention refers to the field of devices of medical use and methods of treatment for correcting malformations, particularly for specific compression of thorax for the purpose of correcting malformations, especially of the type of pectus carinatum.

2. Description of Related Art Including Information Disclosed Under 37 CFR 1.97 and 37 CFR 1.98

Several techniques are employed for the correction of osteocartilaginous growth defects leading to deformations of thoracic cage.

In particular, Pectus carinatum malformation may be surgically corrected, for which metal straps are implanted in the patient anchored to the ribs comprising the affected area.

Alternatively to the above, an external device can be applied that makes it possible to apply a certain pressure that can be regulated for correcting osteocartilaginous growth defects in the thoracic cage, with a possibility of temporary adaptation depending on the evolution of these deformities with the treatment.

This purpose has been practically carried out with a set of mechanical pieces developed for playing certain roles depending on the evolution of the patient being treated.

Specifically, this technique requires that a compression action is done with a force that compresses the deformity and reduces it, preventing decrease of vital pulmonary capacity and therefore making a localized compressive action in predetermined areas has been foreseen by the physician depending on the needs in each case.

Patent AR 041766 B1 refers to a device developed under the shape of a harness, mainly characterized by the fact that it comprises basically a framework, which by way of belt is applied around the thoracic cage at the level corresponding to the deformation to be treated. The device has got corresponding suspension means of the patient's shoulders, and comprises two arcs opposes to each other by their concavity and that are linked by their ends laterally relative to the thoracic cage, at the first of said ends by a joint by way of a hinge and at the second one by a closure adjustable according to the pressure that is desired to be made on the deformed area of the thoracic cage wall. Said pressure is carried out by a compressing plate located over a front arc of the framework from which it protrudes internally and that is operatively associated to a sensor that measures the pressure in suitable units (for example, Pa, hPa, kg/cm², psi) resulting from the aforementioned adjustment and which is obtained by means of a back arc support, on the patient himself which extending behind the spinal column, in the longitudinal sense thereof, provides a supporting back plate on the latter.

However, the device described shows a series of drawbacks and difficulties that make it perfectible.

i) In fact, the patented device comprises a back support comprising a vertical plate resting on the patient's spine. Patients treated with this device show pain in the region of the spine caused by the pressure made thereon, especially in cases of scoliosis, even in some cases light injuries thereof are caused.

This vertebral support is, additionally, uncomfortable for certain movements such as sitting and bending.

Therefore it is desirable to remove this back support and to achieve at the same time an effective correcting treatment.

ii) The device of AR 0'41786 B1 shows one single regulation in its conformation and it is that corresponding to the location of the back support. Therefore, a linking piece is available between the two that constituted the back arc. By regulating that plate the center of said support is achieved to be displaced, which is foreseen for cases of additional deformations wherein the center of the sternum does not coincide with the column axis.

Additionally, in compressing the deformity, the patient's thorax widens and it also widens during the treatment due to patient's own development.

Therefore it is desirable to have a device that is more versatile and that allows to make other regulations, i.e. that it is expansible. For example, a device comprising several pieces in an arc connected to each other would allow the patient to continue with the same device during the entire treatment adjusting it as his own physical development and that of the deformation evolve.

iii) In known devices, the front plate makes pressure on the deformity in the thorax whereby the effectively applied pressure required to make the correction is determined by an electronic meter comprising a suitable sensor. Additionally, the sensor comprising one single pressure cell is incorporated in the plate, whereby the plate has a significant thickness for supporting the electronic sensor meter which turns it uncomfortable for the patient.

Since the sensor is incorporated, it is exposed to various external factors such as ambient humidity, patient's sweat, knocks that it could involuntarily suffer in everyday use, etc., therefore measuring can vary and is unreliable.

Therefore, it is desirable to have a measuring device with an external and detachable sensor such that it is only used by the physician or his assistant when measuring pressure is required, inserting it in a front plate using anchorages to that effect for the purpose of getting much more reliable measurings.

Additionally, it is desirable to be able to read the pressure value on a digital screen (“display”) and additionally to have the possibility of sending the data obtained in a wireless way to an interface which in turn is connected to a computer that receives the information in order to process it with a suitable software.

iv) Additionally, it has been noticed that some patients do not use the device for the required time at the pressure that is suitable for an effective treatment, thus the deformity is not effectively treated.

To that effect, it would be appropriate to have a sufficiently compact measuring equipment that comprises not only a more reliable pressure meter, but also at least one suitable sensor for detecting the patient's body temperature and that allows to determine how much time is the patient effectively under the pressure selected by the physician with an associated time-measuring circuit, that is to be able to know for how much time such pressure was effectively made, storing the recorded values in a memory to be later transferred to a computer comprising a suitable program for evaluation thereof.

In this way, the physician will be able to suitably evaluate the results of the treatment on a solid base of experimental data obtained while using the device.

v) On the other hand, making a compression action with a suitable and sufficient force for compressing the deformity and reducing it, must not only avoid a decrease in vital pulmonary capacity in making a localized compression where it is required, but it must also prevent causing a skin injury in the region to be compressed.

Therefore the belt of the device and particularly the compression plate are required to be coated by suitable protections in all those regions that contact the patient's skin avoiding painful injuries and ulcerations.

Therefore, a device is proposed according to what has been set forth above that involves as an integral part thereof all the elements required for obtaining by a simple adjusting action among the same and totally independently from any means other than the device itself, a suitable compression of the deformed region of the thoracic cage wall until effective and final correction thereof is achieved.

This has been achieved with a maximal degree of practicality by the device that is the object of this invention in the form of a harness or similar, which can be easily applied to the patient being treated by simply suspending it from his shoulders and adjusting it around his thoracic cage, purely and exclusively on the deformation intended to be treated, without support on the patient's spinal column, embracing it only at a height according to the location of said deformation. Thus, all the rest of the thoracic wall is left free and consequently without creating any restriction on the vital capacity of the respiratory tract; wherein such device can be expansible depending on the evolution of the treatment and on the patient's physical development; and comprising an external and reliable measuring system.

vi) Additionally, it is also desirable to have a system that allows to make all the operations required to adapt the expansible device of medical use for specific compression of thorax to the patient's needs when evaluating him, avoiding delays and innumerable fitting sessions for the device to be duly adjusted and calibrated. At the same time, the system permits suitable attendance of those patients who live far from large urban attendance centers.

Thus, a system having all these features should comprise:
an expansible device of medical use for the specific compression of the thorax comprising the aforementioned improvements, comprising a series of different lengths and curvatures which can be exchanged until they form the belt of a compressing device meant for a given patient;
a pressure-measuring apparatus with suitable sensors for determining the suitable compression pressure for the malformation to be treated and which at the same time can be inserted in the compressing plate of the expansible device for controlling the pressure made in situ by the expansible device during use by the patient;
an apparatus for measuring the patient's thorax dimensions;
an apparatus for bending the straps that constitute the expansible device in order to adapt them to the patient's thorax profile; and
additionally, a portable computer comprising at least one computer program (software) suitable for evaluation and follow-up of the patient's evolution.

Eventually, the computer can be installed in the physician's consulting room and must comprise at least one suitable computer program.

The different components of the system such as the apparatus for measuring thorax dimensions, the pressure-measuring apparatus with sensors ad hoc and the strap-bending apparatus are preferably portable.

### BRIEF SUMMARY OF THE INVENTION

Therefore, one object of this invention is an expansible device of medical use for specific compression of thorax comprising a framework applicable in the form of a belt around a patient's thoracic cage comprising two arcs, one front and another back relative to the thoracic cage, both arcs facing each other by the concave internal side and connected to each other on both sides of the thoracic cage by means of a joint at the first ends of both arcs consisting of a turning axis hinge perpendicular to the plane of the belt and that can allow to open them by the second ends of such arcs whereby they are connected by an adjustable side closure. The front arc has a plate that compresses the deformity being treated placed internally relative to the arc and fixed thereto by suitable fixing means. The device includes suspension means that can be regulated from the patient's shoulders that can keep both arcs spaced relative to the body of the latter resting on the shoulders, said suspension means being a pair of wide belts that can be regulated that are lifted relative to their anchorage in the back arc and passing over the patient's shoulders go down in a parallel and spaced way connecting directly to the front arc. The belt of the device comprises the connection to at least curved six plates. The front and back arcs comprise each at least three curved plates the ends of which overlap connecting the plates to each other by adjustable fixing means and because said compressing plate comprises anchorages in order to detachably associate a measuring equipment with sensors that record the pressure made resulting from adjusting between the second ends of both arcs.

In a preferred embodiment of this invention, the front arc of the device comprises three front curved plates and the back arc thereof comprises three back curved plates.

The three front curved plates are the right front plate (PDD), a the middle front plate (PDC) and the left front plate (PDI).

The three back curved plates are the right back plate (PTD), the middle back plate (PTC) and the left back plate (PTI).

Preferably, the PDD has a length between 7 and 10 cm, the PDC has a length between 16 and 32 cm, the PDI has length between 7 and 12 cm, the PTD has a length between 7 and 10 cm, the PTC has a length between 16 and 32 cm, and the PTI has a length between 7 and 12 cm.

The sizes of the plates corresponding to a patient's thorax size are obtained by relating the correcting pressure, the thorax circumference before correction and the deformity height, which determine the belt sizes.

Preferably, the width of the front and back curved plates is between 30 and 40 mm.

The adjustable fixing means connect in a rigid and removable way the device plates by means of screws applied in cooperating rows of holes that can allow to vary the coupling distance between said plates and concomitantly the belt diameter.

More preferably, the device comprises a closure between the second ends of both arcs, created by means of respective flexible tabs connected by an adjustable closure by a latch for preventing involuntary opening thereof.

The front and back plates of the belt of the device of this invention are made from aluminum or plastics laminated with pressure-resistant fibers.

Preferably, the front and back plates of the belt internally comprise protections. Said protections are selected from cushions, inflatable pads, silicone pads and combinations thereof. Preferably, the materials used in making the protections are hypoallergenic. More preferably, the materials of the protections do not comprise latex.

A compact measuring apparatus attached to the compression plate in use of the device by the patient which comprises a pressure meter, at least one suitable sensor for sensing the patient's body pressure and an associated time-measuring circuit in order to determine for how much time the patient is effectively subject to the pressure selected by the physician, recording the values in a memory to be transferred to a computer comprising a suitable program for evaluation thereof.

In a preferred embodiment, the pressure meter averages the values obtained by at least four sensors consisting of interconnected loading cells (“strain gage”) that convert the reading made by the same to pressure values by means of an electronic converting circuit.

Preferably, the measuring equipment comprises a digital screen for displaying the recorded values.

Preferably, the recorded values are sent by an interface, wherein the interface is connected to a computer that receives said value and processes it via an appropriate software. Additionally, the recorded values can be sent to an interface in a wireless way.

The pressure measuring apparatus with suitable sensors for determining the effective compression pressure for compressing the deformity to be treated and which at the same time can be inserted in the compressing plate of the expansible device of medical use for thorax specific compression in order to control in situ the pressure made by the expansible device while being used by the patient.

In a preferred embodiment, the pressure measuring apparatus averages the values obtained by at least four sensors consisting of interconnected loading cells (“strain gage”) which convert the reading made by them to pressure values by means of an electronic converting circuit.

Preferably, the pressure measuring apparatus allows, additionally to reading the pressure value on a digital screen, sending said value to an interface in a wireless way, wherein the interface is connected to a computer that receives and processes said value via at least one appropriate computer program (software).

Preferably, each pressure sensor determines values ranging from 0 to 10.4×10⁻⁴ Pa (from 0 to 15 psi).

In order to achieve an effective treatment, the pressure made by the belt on the protrusion is the same or lower than 172.4 kPa (2.5 psi).

A portable system useful in the treatment of a thoracic deformity, comprising:
an expansible device of medical use for thorax specific compression comprising the aforementioned improvements, comprising a series of plates of different lengths and curvatures which can be exchanged until they form the belt of a compressing device meant for a given patient;
a pressure-measuring apparatus with suitable sensors for determining the suitable compression pressure for the malformation to be treated and which at the same time can be inserted in the compressing plate of the expansible device for controlling the pressure made in situ by the expansible device during use by the patient;
an apparatus for measuring the patient's thorax size;
an apparatus for molding/bending the straps that form the expansible device in order to adapt them to the patient's thorax profile; and additionally,
a portable computer comprising at least one computer program (software) suitable for the evaluation and follow-up of the patient's evolution.

Eventually, the computer can be installed in the physician's consulting room and must comprise at least one suitable computer program.

An apparatus for measuring a patient's thorax size comprising a graduated ruler and a cursor sliding thereon wherein the length of the distance between the ends of both the ruler and the cursor is indicated by the latter on the former.

In another embodiment, the thorax size can be obtained by a cursor comprising a spherical or circular sliding element associated to a electronic circuit which, in describing the perimeter to be determined, allows to obtain the values required to suitably size the belt of the expansible device of medical use for thorax specific compression.

An apparatus for molding/bending straps is an electromechanical device comprising a motoreducer that pulls a shaft ending in a wedge acting on a space between the two facing fixed stops, wherein said wedge presses on a strap placed resting on the aforementioned stops for modifying their curvature radius and adapting it to the patient's needs.

All the components of the system, especially the apparatus for measuring the thorax size, the pressure measuring apparatus with sensors ad hoc and the apparatus for bending the straps, are portable.

### BRIEF DESCRIPTION OF THE SEVERAL VIEWS OF THE DRAWINGS

In order to make understanding of this invention easier and to make possible a better understanding of its scope, the device or harness that is the object of this invention has been illustrated in several figures, everything by way of example.

FIG. 1 is a front perspective view of a device of medical use for thorax specific compression according to one of its preferred embodiments of the prior art, which is shown detached from the patient's shoulders, showing the general arrangement of the component elements.

FIG. 2 is a front top perspective view of the expansible device of medical use for thorax specific compression according to a preferred embodiment of this invention, which is shown detached from the patient's shoulders, showing the general arrangement of the component elements.

FIG. 3 is a back top perspective view of the device in FIG. 2 above.

FIG. 4 is a top side perspective view of the device in FIG. 2 above.

FIG. 5 is a top plan view of the scheme of the distribution and interrelation of the component plates of an embodiment of the expansible device of medical use for thorax specific compression according to this invention.

FIGS. 6A and 6B show perspective views of another preferred embodiment of the device of medical use for thorax specific compression, wherein the compression plate comprises a compact measuring apparatus attached thereto comprising an electronic pressure measuring equipment, an electronic temperature measuring equipment, a time measuring circuit and a memory for storing recorded values.

FIG. 7 (A) shows the front elevation view and FIG. 7 (B) shows the back perspective view of a face of a preferred embodiment of a pressure measuring apparatus with sensors suitable for determining the effective compression pressure for compressing the deformity to be treated, which at the same time can be inserted in the compressing plate of the expansible device of medical use for thorax specific compression for controlling in situ the pressure made by the expansible device while being used by the patient.

FIG. 8 shows a perspective view of a preferred embodiment of the apparatus for measuring a patient's thorax size comprising a graduated ruler and a cursor sliding thereon, wherein the length of the distance between the ends of both the ruler and the cursor, is indicated by the latter on the former.

FIG. 9 shows a perspective view of a preferred embodiment of the portable strap-bending apparatus that is an electromechanical device comprising a motoreducer that pulls a shaft ending in a wedge that acts on a space between two facing fixed stops.

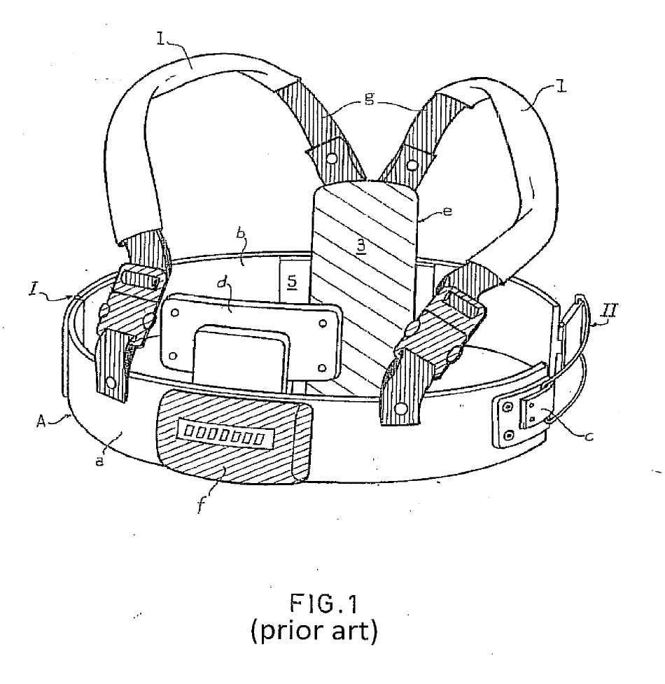

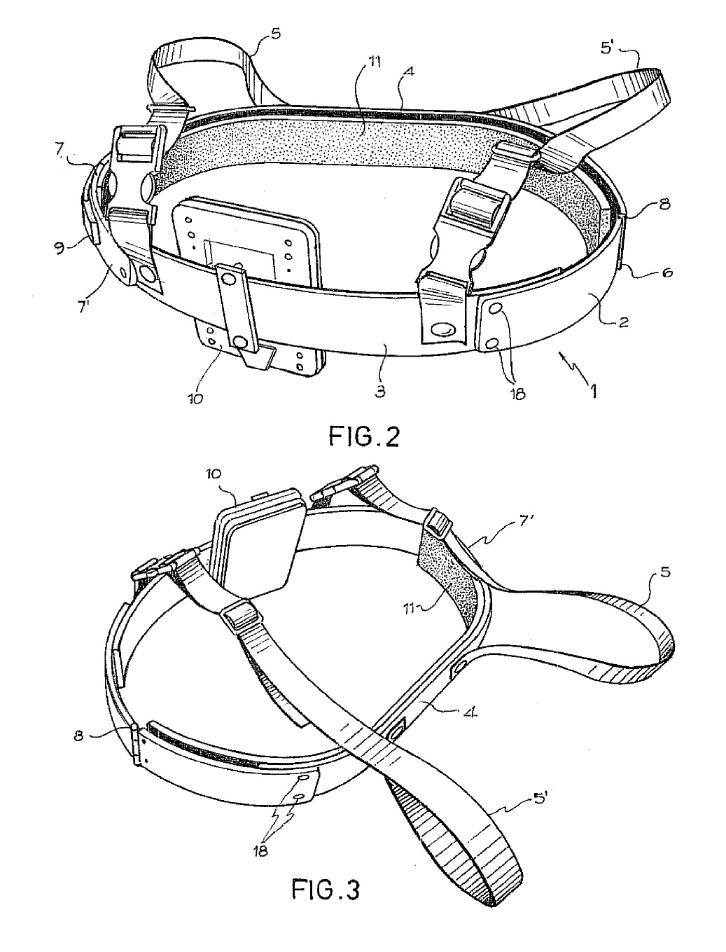

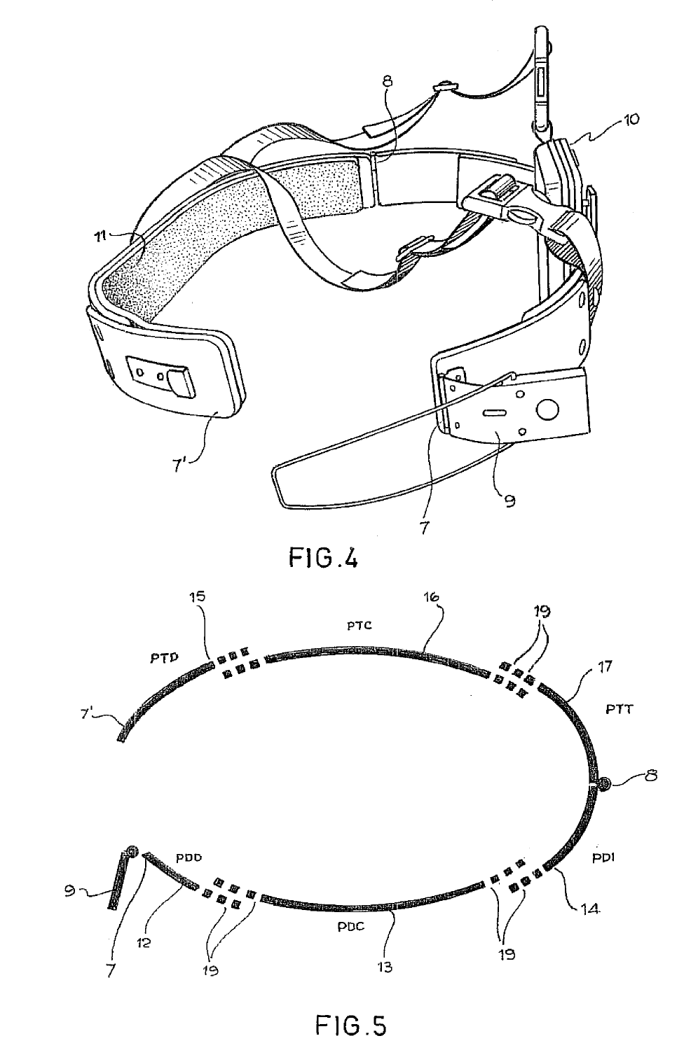

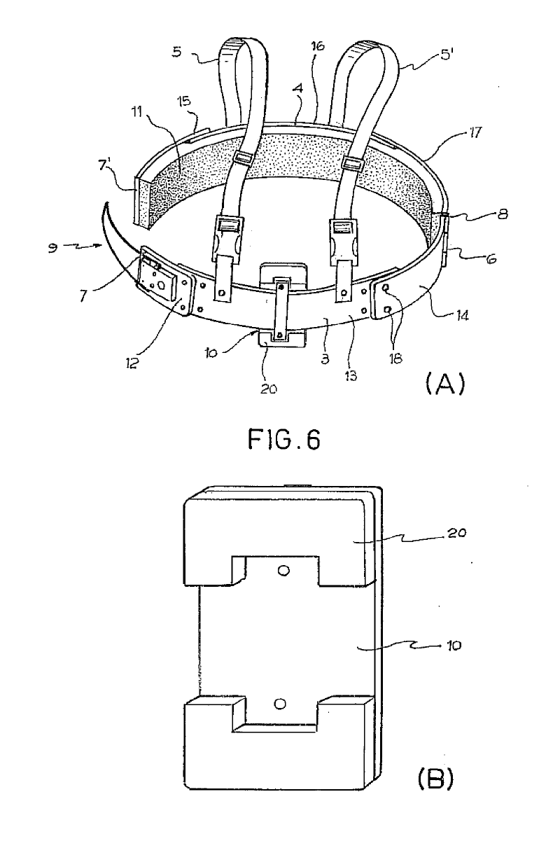

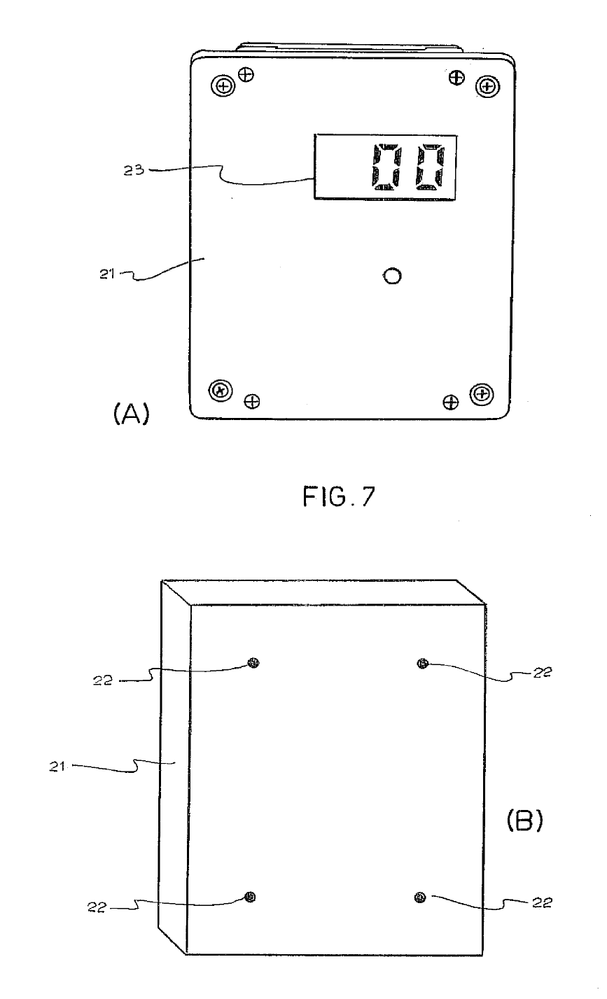

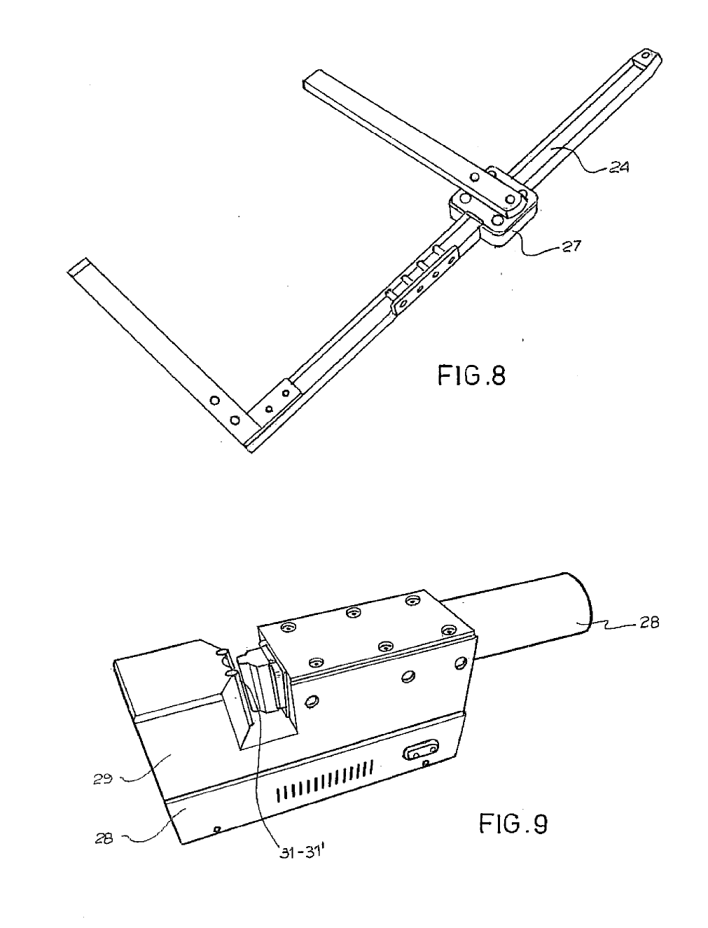

In the Figures, like references indicate similar or corresponding elements or parts.

### DETAILED DESCRIPTION OF THE INVENTION

As indicated previously, it is one object of this invention to provide an expansible device (1) of medical use for thorax specific compression which is applied on and around a patient's thoracic cage under conditions that make it possible to regulate their positioning and the pressure that is to be made thereby on osteocartilaginous deformations due to growth defects, especially those known as pectus carinatum, using their components themselves.

This expansible device (1) of medical use for thorax specific compression comprises a framework that supports the means whereby the compressing action is to be made and, basically, comprises a belt (2) comprising two sections, a front one (3) and a back one (4), faced by their concave internal side, said sections (3, 4) being preformed similarly to the basic virtual configuration of the cross section of a patient's thoracic cage around which they are to be suspended jointly from the patient's shoulders by wide belts (5, 5′) that can be regulated according to and for the purposes that are explained later.

Both sections of the belt (3, 4) shall be designated herein below, for the purposes of a better understanding, as the front arc (3) and back arc (4), which are connected to each other by their adjacent ends (6, 7 and 7′) to be laterally located relative to the patient's thoracic cage, i.e. at either side thereof.

At the first end (6), this connection is created in an articulated way by a hinge (8), the turning shaft of which, preferably a pin passing through the respective articulated plate hole (14 and 17), is oriented perpendicular to the plane on which both arcs (3, 4) extend and are articulated. Everything is to be able to open the belt (2) in order to make their application around the patient's thoracic cage easier. While at the second end (7, 7′), this connection is achieved by means of an adjustable closure (9) which, in general, can be made according to any of the arrangements foreseen by the art regarding fastening that can be regulated between both ends of a belt. Since a relevant matter, as is the fact that the pressure that is applied on the deformity, although it is the result from the side adjustment referred to above between the second ends (7, 7′) of one (3) or another arc (4), is not made directly through the aforementioned connections, but this happens indirectly on the section of the thoracic cage wall being treated by the compressing plate (10) acting directly on the deformity, which is fixed to the front arc (3) lightly protruding from their inner side.

Thus, it is about achieving a compressing action strictly localized on the region embraced by the compressing plate (10) with direct incidence on the protrusion or deformity of the patient's thoracic cage wall. In this way any undue pressure that could be cause respiratory dysfunctions is avoided.

On the other hand, the adjustable connection between both arcs (3, 4) centralized on one single side section of the aforementioned belt (2) of the device (1) allows to create an even continuity along its entire contour so that the adjusting action made between their connecting single outer ends (6, 7, 7′) is translated practically into a perfect localization of the pressure applied on the treatment region through said compressing plate (10) without running any risk of destabilizing the belt (2) relative to the supporting points on the patient's thoracic cage.

The size and position of the compressing plate (10) can be adapted specifically to the basic characteristics of the deformation of the thoracic cage to which the expansible device (1) is applied.

The expansible device (1) according to this invention does not comprise a back plate on the spine like the prior art device. Thus, the device (1) described herein makes a distributed pressure on the patient's entire rib arc, whereby it comprises necessarily an effective cushion (11), especially on their back portion whereto an additionally protecting cushion layer is added.

Removal of the back plate tolerates a better adaptation of the patient to the belt (2) and the pains that are found with the prior art device are avoided.

In the preferred embodiment of this invention, the belt (2) of the device (1) of this invention comprises the connection of at least six bent plates (12, 13, 14, 15, 16, 17), wherein the front (3) and back (4) arcs forming the belt (2) comprise each at least three bent plates (12, 13, 14 y 15, 16, 17) the ends of which overlap, the plates (12, 13, 14, 15, 16, 17) are connected to each other by adjustable fixing means (18).

The aforementioned ends of the plates (12, 13, 14, 15, 16, 17) that constitute the front (3) and back (4 arcs have sets of complementary holes (19) which obviously allow to vary the width of the arc of the belt (2) closing or opening it depending on the needs of each case and depending on the evolution of the treatment.

The plates (12, 13, 14, 15, 16, 17) form the arcs (3, 4) being rigidly connected by sets of screws (18), for example of Allen type, so that they keep the structural continuity of the belt (2). At least two pairs of sets of screws (18) re preferably used by each connection, wherein each pair is disposed to fix each of the ends of the plates (12, 13, 14, 15, 16, 17) which overlap each other.

The structure of the belt (2) of the device (1) of this invention permits regulations in every plate (12, 13, 14, 15, 16, 17) that comprise them. These regulations allow to adapt the shape and size of the belt (2) to a given patient and then, during the treatment, to adapt it accompanying his physical development and the evolution of the protrusion by compression, in such a way that with the same and single expansible device (1), the patient completes his treatment integrally.

This is required since, when the treatment starts to be effective, the thorax deformation is corrected causing thoracic widening. For that reason, the shape of the belt (2) must be changed in order to compensate for the changes in the thorax that has occurred, since the front back diameter is reduced and the side diameter is widened.

On the contrary, taking into account that the treatment is applied to children at growth age, should the patient grow, the belt (2) must be widened in order to avoid an excessive compression.

These regulations allow to use the same device (1) during the entire treatment that extends until growth is completed. If eventually growth is excessive, the plates (12, 13, 14, 15, 16, 17) of the belt (2) can be changed by longer ones and compensate for the patient's development.

In the preferred embodiment of the expansible device (1) of this invention, the front arc (3) comprises three front bent plates (12, 13, 14) and the back arc (4) comprises three back bent plates (15, 16, 17). The three front bent plates (12, 13, 14) are the right front plate or PDD (12), the middle front plate or PDC (13) and the left front plate or PDI (14). The three back bent plates (15, 16, 17) are: the right back plate or PTD (15), the middle back plate or PTC (16) and the left back plate or PTI (17).

Preferably, the PDD (12) has length between 7 and 10 cm, the PDC (14) has a length between 16 and 32 cm and the PDI (14) has a length between 7 and 12 cm.

Also preferably, the PTD (15) has a length between 7 and 10 cm, the PTC has a length between 16 and 32 cm and the PTI (17) has a length between 7 and 12 cm.

The sizes of the plates (12, 13, 14, 15, 16, 17) corresponding to a given size of a patient's thorax are obtained through a mathematical formula that as a function of pressure correction, the circumference of the thorax before the correction and the height of the protrusion, determines the size of the belt (2). Said formula is:

MC=MTA+5−( 7/4AP−2)(10−p/6891) 1/9

wherein,
MC is the size of the belt (2) expressed in cm;
MTA is the thorax size taken at the height of the protrusion expressed in cm;
AP is the height of the protrusion expressed in cm; and
p is the pressure applied expressed in Pa; and
wherein, the sizes of each of the plates (12, 13, 14, 15, 16, 17) result from a possible combination that allows to achieve the size of the belt (2) MC calculated. This possible combination of the plates (12, 13, 14, 15, 16, 17) can be obtained from tables or by means of a computer program (software) that allows to obtain the best combinations for each MC.

Preferably, the width of the front (12, 13, 15) and back plates (15, 16, 17) is between 30 and 40 mm.

In a preferred embodiment, adjustable fixing means (18) connect in a rigid and removable to the plates (12, 13, 14, 15, 16, 17) at their ends by means of screws (18) applied to cooperating hole rows (19) that can allow to vary the coupling distance between said plates (12, 13, 14, 15, 16, 17) and concomitantly, the diameter of the belt (2).

Preferably, while the device (1) is being used by the patient, the compressing plate (10) comprises a compact measuring apparatus (20) mounted thereon that comprises an electronic pressure measuring equipment. An electronic temperature measuring equipment associated with at least one sensor is suitable for detecting the patient's body temperature, and an associated time measuring circuit, in order to determine for how much time the patient, is effectively under the pressure selected by the physician, storing the recorded values in a memory to be transferred to a computer comprising a program suitable for that evaluation.

In this preferred embodiment, the electronic pressure measuring equipment averages the values obtained by at least four sensors consisting of interconnected loading cells (“strain gage”) that convert the reading made by them to pressure values by means of an electronic converting circuit.

In the preferred embodiment, the measuring equipment (20) comprises a digital screen for displaying the values recorded.

Preferably, the recorded values are sent to an interface, wherein the interface is connected to a computer that receives said value and processes it via an appropriate software. Additionally, the recorded values can be sent to an interface in a wireless way.

Additionally, through a pressure measuring apparatus (21) with suitable sensors, the physician determines the effective compression pressure for compressing the deformity to be treated. At the same time, this pressure measuring apparatus (21) can be inserted in suitable anchorages in the compressing plate (10) of the expansible device (1) of medical use for the thorax specific compression in order to control in situ the pressure made by said expansible device (1) while being used by the patient as a result of the closure between the second ends (7 and 7′) of both arcs (3, 4).

The pressure measuring apparatus (21) is independent from the expansible device (1) and different from the compact measuring equipment (20) that can be mounted on the compressing plate (10). This apparatus (21) is normally found in the physician's hands. When measuring pressure is required, it is inserted in the compressing plate (10) by using the anchorages comprised therein for that purpose. In this way, they can achieve reliable measurements and, in turn, the measuring device can be calibrated against possible failures.

Consequently, the thickness of the compressing plate (10) is considerably reduced, which means that it is not noticed under the patient's clothes.

The prior art device comprised a pressure measuring apparatus with one single sensor connected to the single pressure cell. This means that the value measured varied depending on where and how pressure was made on the compressing plate.

Whether it is used independently or that it is fixed at the corresponding anchorages of the compressing plate (10), the pressure measuring apparatus (21) comprises at least four sensors (22) consisting of interconnected loading cells (“strain gage”) that provide and/or record the pressure resulting from directly compressing the protrusion during diagnosis or the pressure caused by adjustment between both second outer ends (7 and 7′) of the belt (2). Said sensors (22) provide pressure amount data by measuring it in suitable units (for example, Pa, hPa, kg/cm2, psi) so as to make only the required pressure, no more no less, required as a function of the degree of correction that is sought to be obtained at each treatment session. The pressure reading is obtained by means of an electronic circuit which converts the readings of the sensors (22) as an average of the readings of all the sensors (22) distributed throughout the surface of the compressing plate (10), thereby providing a value that is much more adjusted to reality estimating that the values that are obtained are at least 95% reliable.

The pressure measuring apparatus (21) preferably allow to display the pressure value on a digital screen (23), for example a liquid crystal display, wherein the determined pressure value can be seen in the suitable (for example, Pa, hPa, kg/cm², psi) and/or more preferably send such value to an interface in a wireless way, wherein the interface is connected to a computer that receives and processes such value via at least one appropriate computer program (software). This program allows to store all data from patients being treated and additionally different statistics can be obtained regarding the effectiveness of the treatment.

Since the equipment is wireless, it works with power provided by a battery. As its capacity decreases due to its use, measurements might be affected, therefore the measuring equipment comprises a low battery warning for replacement whereof.

Preferably, each sensor (22) can determine pressures ranging from 0 to 10.4×10⁻⁴ Pa (equivalent to 0 to 1.06 kg/cm², or between 0 and 15 psi, being 1 psi=1 pound/square inch), pressures being made once the device has been placed, preferably ranging below about 172.4 kPa (equivalent to 0.176 kg/cm², or 2.5 psi). Should it be required, these preferred ranges can be changed and adapted depending on the patient without changing the scope of this invention.

As already indicated, the expansible device (1) so constituted according to its basic general features, is completed by a suspension means arrangement (5, 5′) developed for the purposes of supporting the device from the belt (2) in such a way that its plates (12, 13, 14, 15, 16, 17) can be arranged at the required height from the time when they are applied to the patient and during all the time that correcting action is made, with the possibility of easily regulating said height while adjustment is carried out until the proper positioning of the compressing plate (10) on the deformity is achieved.

These means (5, 5′) preferably comprise classic traces or wide belts of an adjustable length, preferably with protecting shoulder reinforcements on the patient's shoulders, and both wide belts start, for example, from respective engagements on the back arc (4) or from the ring applied on the middle plate (16) of the back arc and relative to which they open going up the patient's back up to his shoulders from which they go down substantially parallel up to their connection to the front arc (3), on either side of the patient's thorax in such a way that when they are extended they leave a broad free space for proper operation of the compressing plate (10) and the apparatus for measuring the pressure made and the patient's body temperature as a function of time.

Regarding the aforementioned lateral adjustment means between the outer second ends (7 and 7′) of both arcs (3, 4), it has been foreseen, by way of example, to adopt an arrangement of the means equivalent to the classic closure (9) of a belt by a flexible tab with a buckle catch which obviously can be substituted by any other kind of closure (9) which permits an effective control of the tension made between both ends (7, 7′) and, at the same time, a firm removable interlocking at the degree of adjustment determined in each case, so as to make its graduation depending on the evolution of the correction sure and possible.

A closure (9) by engagement between two adjustable buckles at respective flexible tabs at both ends (7, 7′), or a closure (9) with an engagement and tension, could also be used.

As described above, the expansible device (1) of this invention preferably comprises a closure (9) created between the second ends (7 and 7′) of both arcs (3, 4) by respective flexible tabs connected by an adjustable closure (9), comprising, additionally, a latch for preventing its involuntary opening.

Additionally and if required, planetary gearings or additional compressing plates fixed in the belt of the device (1) of the invention by respective sheets that keep them on eventual thoracic deformations that are shown in compressing the primary thoracic malformation that is corrected. The thoracic contour is thereby integrally shaped.

The different front (12, 13, 14) and back plates (15, 16, 17) of the belt (2) of the device (1) according to this invention can be made from any material that allows to apply pressure on a thoracic malformation to be corrected and to keep it constant and effective enough during the entire treatment, allowing the patient to breathe comfortably.

The proper materials for making the aforementioned plates (12, 13, 14, 15, 16, 17) are aluminum, plastics laminated with pressure-resistant fibers, etc., which allow to apply high pressures without deforming.

It is suitable to indicate that the inner side of the plates (12, 13, 14, 15, 16, 17) comprising both arcs (3, 4) like that of the compressing plate (10) are conveniently coated by protections, which are made up of suitable protecting coatings (11) in the sections of the thoracic cage wall and the deformity on which they are contact while using the device (1).

Said internal protections (11) are selected from cushions, inflatable pads, silicone pads and their combinations. Preferably, the materials used in their construction are hypoallergenic. More preferably, the materials of the protections do not comprise latex.

A portable system useful in the treatment of pectus carinatum, comprising:
an expansible device (1) of medical use for thorax specific compression comprising the aforementioned inventive characteristics, comprising a series of plates (12, 13, 14, 15, 16, 17) of different lengths and curvatures that can be exchanged until they form the belt (2) of a compressing device meant for a given patient, wherein the compressing plate (10) comprises mounted thereon a compact measuring apparatus (20) comprising an electronic pressure measuring equipment, an electronic temperature measuring equipment associated with at least one sensor suitable for detecting the patient's body temperature and an associated time measuring circuit for continuously measuring the patient's body temperature during the time that compression force is applied effectively, storing in a memory the values and times recorded;
a pressure measuring apparatus (21) with suitable sensors (22) for determining the suitable compression pressure for the malformation to be treated and that at the same time can be inserted in the compressing plate (10) of the expansible device (1) of medical use for thorax specific compression for compressing the pressure made in situ by the expansible device (1) once applied to the patient;
an apparatus for measuring (24) the patient's thorax size;
an apparatus for molding/bending (25) the straps that form the expansible device in order to adapt them to the patient's thorax profiles; and
additionally, a portable computer comprising at least one computer program (software) suitable for evaluation and follow-up of the patient's evolution.

Eventually, the computer can be installed in the physician's consulting room and that must comprise at least one suitable computer program.

The apparatus for measuring (24) a patient's thorax size comprising the graduated ruler (26) and a sliding cursor (27) thereon wherein the length of the distance between the ends of both the ruler (26) and the cursor (27) is indicated by the latter on the former.

In another embodiment, the thorax size can be obtained by a cursor comprising a spherical or circular sliding element associated to an electronic circuit which, in describing the perimeter to be determined, allows to obtain the values required for suitably sizing the belt (2) of the expansible device (1) of medical use for the thorax specific compression. Additionally, the data can be sent to a computer and are loaded to the program via the same interface. Preferably, the data are sent in a wireless way.

The expansible compressing device (1) of medical use, the compact measuring apparatus (20) mounted on the compressing plate (10) comprising a pressure measuring apparatus, at least one suitable sensor for detecting the patient's body temperature and an associated time measuring circuit, and the pressure measuring equipment (21) of the portable system of medical use in the treatment of thoracic deformations, preferably of pectus carinatum type, are complemented by an apparatus (25) for changing the curvature of the plates (12, 13, 14, 15, 16, 17) that form the belt (2) when required by the circumstances.

Since the apparatus (25) for changing the curvature of the plates (12, 13, 14, 15, 16, 17) is portable, it allows to make corrections of the plates (12, 13, 14, 15, 16, 17) directly in the physician's consulting room during patient's attention rapidly and saving time as required.

This apparatus form molding/bending (25) the plates (12, 13, 14, 15, 16, 17) consists of a motoreducer (28) associated to an electronic circuit allowing to make large forces with the equipment of reduced size.

In effect, the apparatus for molding/bending (25) the plates (12, 13, 14, 15, 16, 17) is an electromechanical device comprising a motoreducer (28) that pulls a shaft ending in a wedge (29) acting on an apparatus (30) that is between two facing fixed stops (31, 31′). The wedge (29) presses the plate (12, 13, 14, 15, 16, 17) arranged resting on said stops (31, 31′) for molding/bending changing its curvature and adapting it to the patient's needs.

All the components of the system, especially the apparatus for measuring the thorax size (24), the pressure measuring apparatus (21) with sensors (22) ad hoc and the bending apparatus (25) of the plates (12, 13, 14, 15, 16, 17) are portable.

Mode of Using the Device

From the operation point of view, the physician determines firstly what pressure must be applied to the deformity to be treated for its correction by the device of the invention. Once it is verified that the deformation is sensitive to the pressure applied, it is also verified whether the correction does not lead to side deformations in making such pressure. The pressure is determined by the corresponding measuring apparatus.

Then, the patient's thorax size is measured by a suitable tool or cursor by loading it on the memory of the measuring equipment and with the pressure values obtained the required adaptations to the device for a particular case to be treated are made.

Once the arc is adjusted to the patient's thoracic contour, the regulation of the height is made by adjusting the wide belts with buckles and tracks for that purpose, positioning the belt compression plate at the height of the deformation to be corrected.

Then, the belt end closure of the device is adjusted in the proper position and the pressure made by the corresponding measuring equipment is verified. The required corrections are made until the suitable pressure on the deformity to be corrected is achieved.

Then, periodical controls are made in order to see the evolution of the correction and to implement the adjustments that may be required as sought results are achieved.

During these evaluations it is also verified for how much time the patient used the device and at what compressing pressure, which is relevant when evaluating the effectiveness of the treatment. This is done by a compact measuring apparatus mounted on the compressing plate comprising an electronic pressure measuring equipment, an electronic temperature measuring equipment associated to at least one sensor suitable for detecting the patient's body temperature and an associated time measuring circuit for continuously measuring the patient's body temperature during the time that the compressing force is effectively applied, storing on a memory the values and times recorded. These temperature values can be derived to a computer having a suitable software.

The measuring apparatus used in obtaining these values is compact and comprises a chip of a memory that allows to store the data obtained of the compressing pressure made, the body temperature and the time during which the pressure made was effective, until they are transferred to a computer. Eventually, the measuring apparatus comprises a screen that allows to see the values accumulated in the memory.

Additionally and should this be required, the curvature of the plates of the belt can be corrected by using a plate molding equipment that is portable and allows an effective response during the patient's visits to the attending physician in order to evaluate the effectiveness of the treatment.

All that has been described herein corresponds to the best mode of carrying out the invention, bearing in mind that variations that are apparent for a person skilled in the art must be considered within the scope of this invention.

We claim:

1. A method for correcting a thoracic deformity in a patient, comprising the steps of:
a) determining the pressure required for the correction;
b) determining whether additional deformations occur in making said correcting pressure;
c) measuring the thorax size;
d) processing the data obtained in b) and c);
e) making an expansible device of medical use for thorax specific compression according to the results obtained in d);
f) applying the expansible device in treatment; and
g) controlling the pressure made by the compressing plate of the expansible device.

2. The method according to claim 1, wherein the pressure made by the compressing plate of the expansible device is the same or below 17.24 kPa (2.5 psi).

3. The method according to claim 1, further comprising the step of
evaluating the effective use time of the expansible device.

4. The method according to claim 3, wherein the evaluating step is based on the data of the pressure effectively made, the body temperature and the time obtained while the expansible device is being used.
